(19) United States
(12) Patent Application Publication
(10) Pub. No.: US 2002/0072068 A1
(43) Pub. Date: Jun. 13, 2002

(54) Spotting pin and device for fabricating biochips

(76) Inventors: Motonao Nakao, Kanagawa (JP); Katsuya Mizuno, Kanagawa (JP); Junji Yoshii, Kanagawa (JP); Akihiro Asal, Kanagawa (JP)

Correspondence Address: Pillsbury Winthrop LLP, Suite 200, 11975 El Camino Real, San Diego, CA, 92130, US

(21) Appl. No.: 10/007,903

(22) Filed: Dec. 6, 2001

(30) Foreign Application Priority Data
Dec. 8, 2000 (JP) 374833/2000

(51) Int. Cl. C12Q 1/68; A61L 2/00; B05D 3/00; C12M 1/34; B43K 5/16

(52) U.S. Cl. 435/6; 427/2.11; 401/260; 435/287.2

(57) ABSTRACT

Provided are a spotting pin capable of sequential and uniform spotting and a device for fabricating biochips using the spotting pin. The spotting pin of the present invention capable of sequential spotting includes an internal hollow tube of a tubular shape, an external tube slidable on an outer surface of the internal hollow tube, a piston of which one end is fixed to said external tube and which is made slidable inside the internal hollow tube, a spring disposed inside the external tube for resisting the force to move the external tube toward a direction of a tip of said internal hollow tube, and a stopper provided in a position of a given distance from the tip of the internal hollow tube.

### PRIORITY INFORMATION

This application claims priority to Japanese Application Serial No. 374833/2000, filed Dec. 8, 2000.

### BACKGROUND OF THE INVENTION

1. Field of the Invention

The present invention relates to a spotting pin for use in fabrication of biochips and to a device for fabricating biochips incorporating the spotting pin.

2. Prior Art

With respect to studies of genes in biochemistry, experiments such as hybridization has heretofore been performed by use of biochips fabricated in a manner that probe sequences composed of plural types of DNA, RNA, DNA fragments, RNA fragments, proteins or other biopolymers are spotted on a substrate made of a glass plate, nylon, a nitrocellulose membrane or the like. FIGS. 13A and 13B are views for describing a conventional method of fabricating a biochip. As shown in FIG. 13A, prepared are: a microplate 102 containing plural types of probe DNA 101; and a glass plate as a substrate 103 for a biochip. Then, the probe DNA 101 contained in the microplate 102 is adhered to a pin 105, and the probe DNA 101 adhered to the pin 105 is allowed to be spotted on the glass plate 103 by contact therewith. Such operations would be iterated until all types of the probe DNA contained in the microplate 102 are spotted, thus fabricating a biochip 110 which is obtained by spotting multiple types of probe sequences 106 on a surface of the plate in accordance with predetermined arrays, as illustrated in FIG. 13B.

FIGS. 14A to 14C are explanatory views of a conventional spotting pin used for fabrication of biochips. The drawings show a cylindrical spotting pin 125 of a stamping type, of which the tip is planar. In the event of spotting the probe DNA, as shown in FIG. 14A, first the spotting pin 125 is put into a cup 122 containing the probe DNA 121 so that its tip picks up the probe DNA 121. Then, as shown in FIG. 14B, the tip of the spotting pin 125 with adhesion of the probe DNA 121 is dabbed on a glass plate 123 or the like, whereby stamping is performed. In this way, a spot 124 of the probe DNA is formed on the glass plate 123 as shown in FIG. 14C.

FIGS. 15A to 15C are views for describing the fundamentals of hybridization using a biochip. As shown in FIG. 15A, a biochip 131 with probe DNA 132 spotted thereon, and sample DNA 133 marked with fluorescent materials 134 are put together in a hybridization solution 135 and are allowed to hybridize. The hybridization solution 135 is a liquid mixture composed of formaldehyde, standard saline citrate (SSC: NaCl, trisodium citrate), sodium dodecyl sulfate (SDS), ethylene diamide tetraacetic acid (EDTA), distilled water and the like. The mixing proportion thereof may vary according to behavior of the DNA used therein. In this event, if the sample DNA 133 and the probe DNA 132 on the biochip 131 are complementary strand DNA's to each other, then the both items form the double helix structure and are thereby bound to each other. On the contrary, they are not bound if they do not possess complementary strands to each other. Thereafter, as shown in FIG. 15B, the sample DNA 133 marked with the fluorescent materials 134 that remains on the glass plate 133 is soaked in water 136 and washed off, whereby the sample DNA not bound to the probe DNA 132 is drained out. Then, as shown in FIG. 15C, the fluorescent materials 134 that are marking the sample DNA bound to the probe DNA are excited by light energy from a lamp 137. Then, detection of hybridization is performed by means of detecting light emitted by excitation of the fluorescent materials, with an optical sensor 138 such as a CCD.

The conventional spotting pin is incapable of spotting on a plurality of substrates (such as glass plates) sequentially. The biochip needs to be formed with several thousands to several tens of thousands of spots thereon. Accordingly, if operations of drawing the pin back to the position of the cup containing the probe DNA and the like, dipping the tip of the pin into the cup and adhering the probe to the tip are iteratively performed in each stamping, such iteration would require enormous time to fabricate the biochip. On the other hand, assuming that sequential spotting on the plurality of substrates is feasible, possible differences in quantity of the probe sequences contained in the spots on each substrate from one another may incur experimental errors in subsequent steps of hybridization and detection.

### SUMMARY OF THE INVENTION

Given the current circumstances of fabrication of biochips as described above, an object of the present invention is to provide a spotting pin capable of spotting uniform spots sequentially and a device for fabricating biochips by use of such a spotting pin.

In the present invention, in order to effectuate sequential spotting, a spotting pin including a syringe and a stopper at the tip of the syringe for enabling smooth pick-ups of a sample solution is developed.

Specifically, a spotting pin of the present invention comprises: an internal hollow tube of a tubular shape; an external tube slidable on an outer face of the internal hollow tube; a piston of which one end is fixed to the external tube, and which is made slidable inside the internal hollow tube; a spring disposed inside the external tube for resisting the force to move the external tube toward the direction of a tip of the internal hollow tube; and a stopper provided in a position of a given distance from the tip of the internal hollow tube.

Another spotting pin of the present invention comprises: an internal hollow tube of a tubular shape; an external tube having a bottom, which is slidable on an outer surface of the internal hollow tube; a spring disposed inside the external tube for resisting the force to move the external tube toward the direction of a tip of the internal hollow tube; and a stopper provided in a position of a given distance from the tip of the internal hollow tube.

Still another spotting pin of the present invention comprises: an internal hollow tube of a tubular shape; an external tube having a bottom, which is slidable on an outer surface of the internal hollow tube; and a stopper provided in a position of a given distance from the tip of the internal hollow tube.

Yet another spotting pin of the present invention comprises: an internal hollow tube of a tubular shape; an external tube slidable on an outer surface of the internal hollow tube; a piston of which one end is fixed to the external tube, and which is made slidable inside the internal hollow tube; and a stopper provided in a position of a given distance from the tip of the internal hollow tube.

The internal hollow tube preferably includes notches on its tip. Such notches are preferably provided in plural in axially symmetric positions of the internal hollow tube.

According to the present invention, provided is a device for fabricating biochips for spotting plural types of probe sequences in predetermined positions on a substrate, which comprises: a substrate stage for placing in alignment a plurality of substrates for fabrication of biochips thereon; a microplate stage for placing a microplate containing the plural types of probe sequences to be spotted; an XYZ driving unit equipped with any one of the foregoing spotting pins and capable of driving a position of a tip of the spotting pin toward the X, Y and Z directions.

According to the device for fabricating biochips equipped with the spotting pin of the present invention, the probe sequences held by suction inside the internal hollow tube can be spotted out accurately on the plurality of the substrates for fabrication of biochips sequentially by constant amounts. Accordingly, mass production of the biochips becomes feasible in a short period of time.

### BRIEF DESCRIPTION OF THE DRAWINGS

FIG. 1 is a schematic cross-sectional view showing one example of a spotting pin according to the present invention.

FIG. 2 is a partial diagrammatic view showing an example of a form regarding a tip of an internal hollow tube.

FIGS. 3A to 3D are views for describing a pick-up operation of a sample with the spotting pin.

FIGS. 4A to 4D are views for describing another pick-up operation of a sample with the spotting pin.

FIGS. 5A and 5B are schematic cross-sectional views for describing another example of a spotting pin according to the present invention.

FIGS. 6A and 6B are views for describing spotting of a sample.

FIG. 7 is a schematic diagram of a device for fabricating biochips according to the present invention.

FIG. 8 is an explanatory view showing one example of a Z-axis driver of an XYZ driving unit.

FIG. 9 is a view showing the XY coordinate system of a stage of the device for fabricating biochips.

FIG. 10 is a view showing the YZ coordinate system of a stage of the device for fabricating biochips.

FIG. 11 is a view showing a method of spotting a sample on a substrate for a biochip while avoiding contact with the substrate.

FIG. 12 is a view showing the XY coordinate system of a dispensing stage.

FIGS. 13A and 13B are views showing a conventional method of fabricating biochips.

FIGS. 14A to 14C are views showing a conventional spotting pin.

FIGS. 15A to 15C are views for describing the fundamentals of hybridization using a biochip.

### DETAILED DESCRIPTION OF THE PREFERRED EMBODIMENTS

Now, embodiments of the present invention will be described with reference to the accompanying drawings.

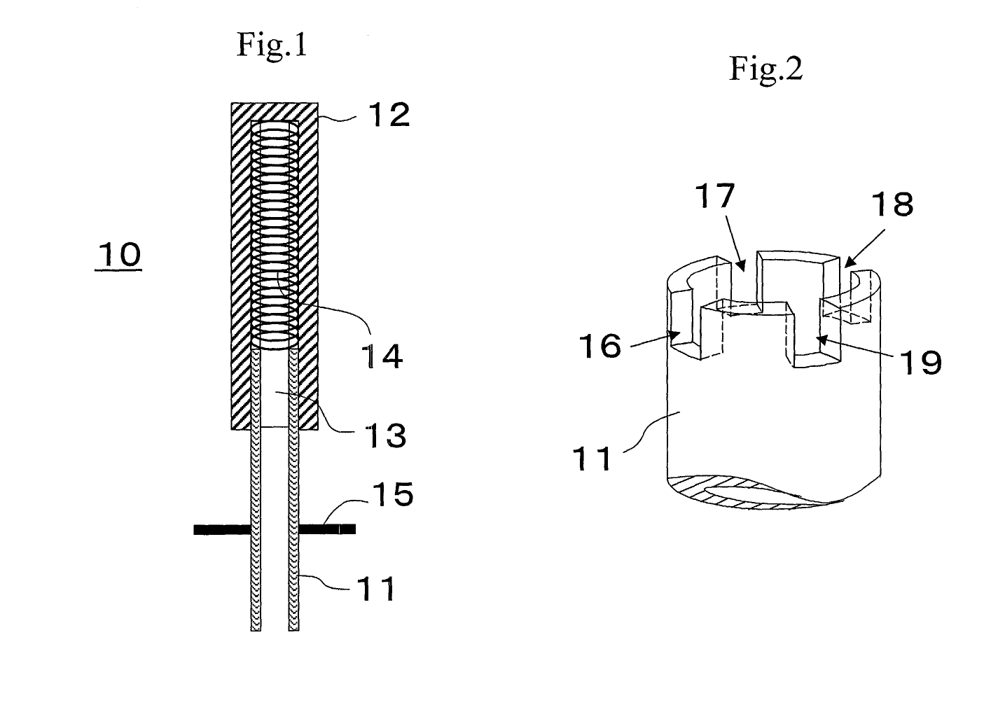

FIG. 1 is a schematic cross-sectional view showing one example of a spotting pin according to the present invention. In this example, a spotting pin 10 has a syringe structure and it comprises: an internal hollow tube 11 of a tubular shape; an external tube 12 slidable on an outer surface of the internal hollow tube; and a piston 13 of which one end is fixed to a bottom of the external tube, the piston 13 which has an external diameter approximately equal to an internal diameter of the internal hollow tube 11 and is fitted into a hollow part of the internal hollow tube 11 so that it is made slidable therein. In a gap between the external tube 12 and the piston 13, a coil spring is inserted therein in a manner that one of its ends is fixed to the bottom of the external tube 12 and the other end is fixed to an end of the internal hollow tube 11. Moreover, the internal hollow tube 11 includes a flanged stopper 15 disposed in a position of a given distance from a tip thereof.

When the external tube 12 is allowed to slide on the outer surface of the internal hollow tube 11 in the direction of the stopper 15, the piston 13 contacts closely with an inner surface of the internal hollow tube 11 and slides thereon while contacting closely therewith. In this event, the spring 14 is pressed between the bottom of the external tube 12 and the end of the internal hollow tube 11. When the force to push the external tube 12 toward the direction of the stopper 15 is released, the external tube 12 and the piston 13 inside go back to the original positions by an action of the spring 14.

FIG. 2 is a partial diagrammatic view showing an example of a form regarding the tip of the internal hollow tube 11. A plurality of notches 16 to 19 are provided at the tip of the internal hollow tube 11, so that the inside of the internal hollow tube 11 and the outside thereof communicate with each other through the notches 16 to 19 even in the event of spotting when the internal hollow tube 11 is blocked by its tip hitting a plane. Accordingly, a fluid (a sample) held inside the internal hollow tube 11 can escape outside through the notches 16 to 19, when, as described later, the tip of the internal hollow tube 11 is pressed to a glass slide or the like and the inside thereof is pressurized. Although shapes or the number of the notches are not particularly limited, it is preferable that plural notches are provided in position axially symmetric with respect to the central axis of the hollow tube 11, because the fluid (the sample) inside the tube is expected to spread uniformly in many directions when the pin is pressed to the plane.

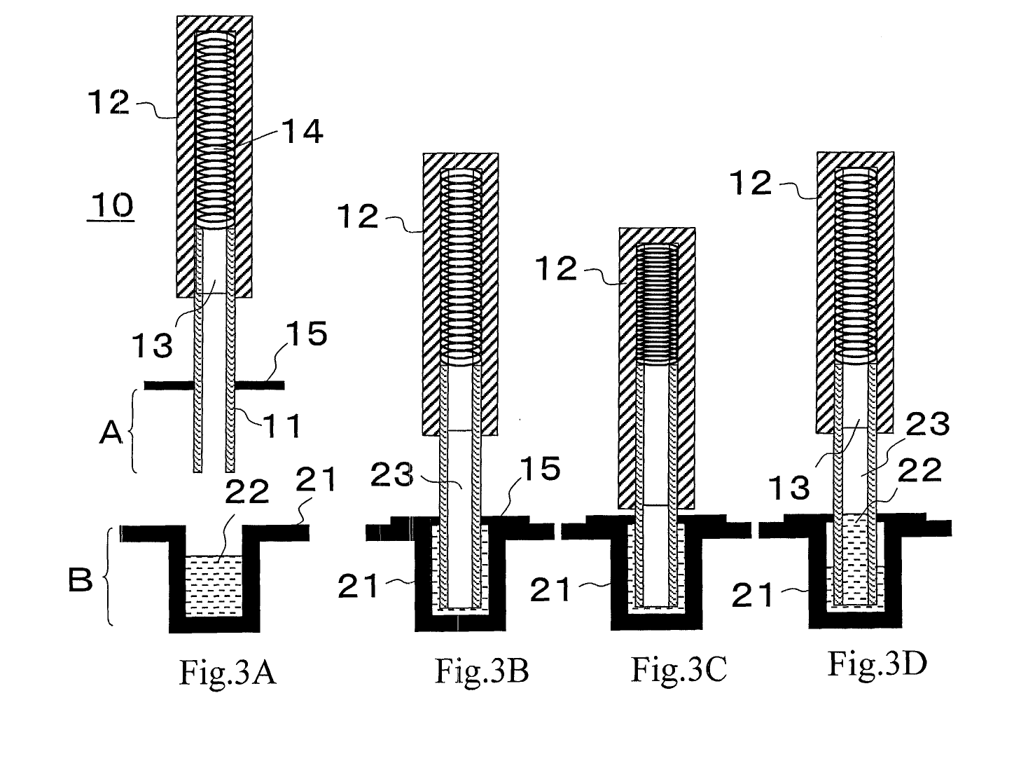

FIGS. 3A to 3D are views for describing an aspect of picking up probe sequences contained in a sample cup (hereinafter referred to as the sample) with the spotting pin shown in FIG. 1. A distance A from the tip of the internal hollow tube 11 to the stopper 15 of the spotting pin 10 is made shorter than a depth B of a sample cup 21 (B>A), whereby the spotting pin 10 is constituted in a manner that the tip of the internal hollow tube 11 of the spotting pin 10 does not touch to a bottom of the sample cup 21, when the spotting pin 10 is pushed into the sample cup 21.

FIG. 3A shows a state that the spotting pin 10 is placed directly above the sample cup 21. A liquid sample 22 is contained in the sample cup 21. Next, as shown in FIG. 3B, the tip of the spotting pin 10 (the internal hollow tube 11) is pushed into the sample cup 21 until the stopper 15 contacts with the sample cup 21. In this event, the inside of the internal hollow tube 11 is filled with air 23. Furthermore, the external tube 12 is pressed downward as shown in FIG. 3C, and the piston 13 is pressed to the inside of the internal hollow tube 11 to evacuate the air inside the internal hollow tube 11. Next, the external tube 12 is pulled up as shown in FIG. 3D. Then, the piston 13 is elevated inside the internal hollow tube 11 by an action of the spring 14 in a state that the internal hollow tube 11 contacts with the sample cup 21 and rests thereon, whereby the sample 22 is drawn into the internal hollow tube 11. An air layer 23 exists between the sample 22 and the piston 13.

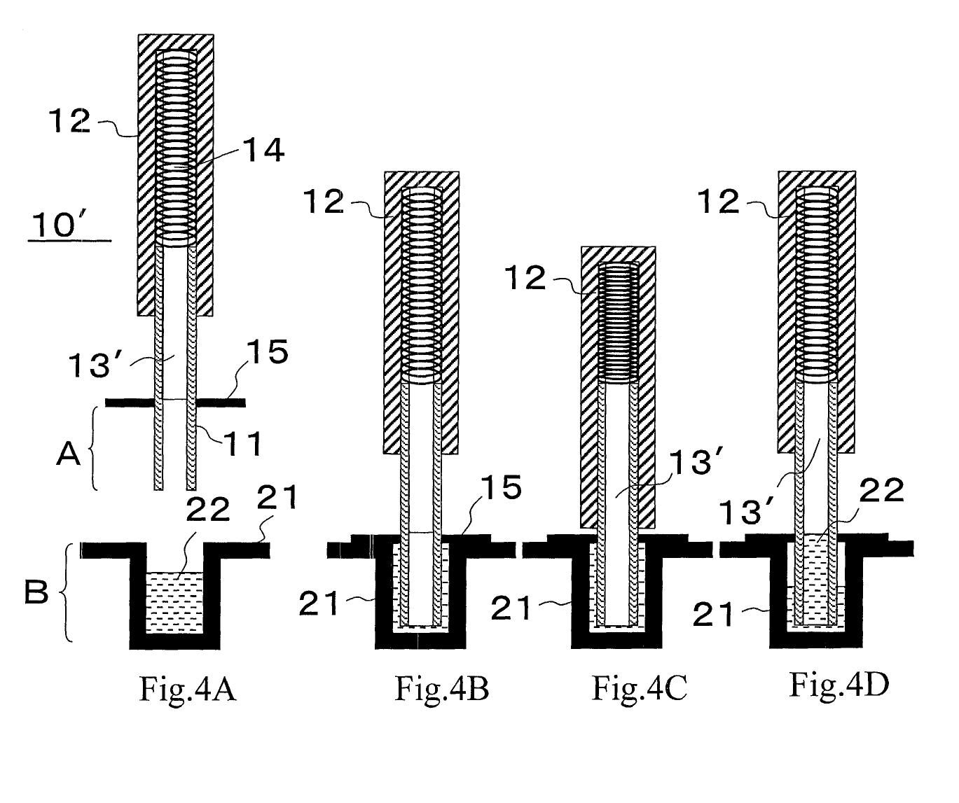

FIGS. 4A to 4D are views for showing a spotting pin 10′ with an elongated a piston and an aspect of suction of the sample by the spotting pin 10′. FIGS. 4A to 4D are views of the states corresponding to those in FIGS. 3A to 3D, respectively. The spotting pin 10′ includes a piston 13′, which is longer than that in the spotting pin 10 shown in FIGS. 3A to 3D. For this reason, air inside an internal hollow tube 11 is entirely evacuated in the state of FIG. 4C that corresponds to the state in FIG. 3C. Accordingly, as shown in FIG. 4D, an air layer 23 does not exist between a sample 22 aspired into the internal hollow tube 11 and a piston 13′, but the sample 22 directly touches to the piston 13′.

It should be noted that the piston is not always necessary. Specifically, if the space between the outer surface of the internal hollow tube 11 and the inner surface of the external tube 12 secure sufficient airtightness, then suction or discharge of the sample in and from the inside of the internal hollow tube 11 becomes feasible just by pushing or pulling the external tube 12 with respect to the internal hollow tube 11 without provision of the piston.

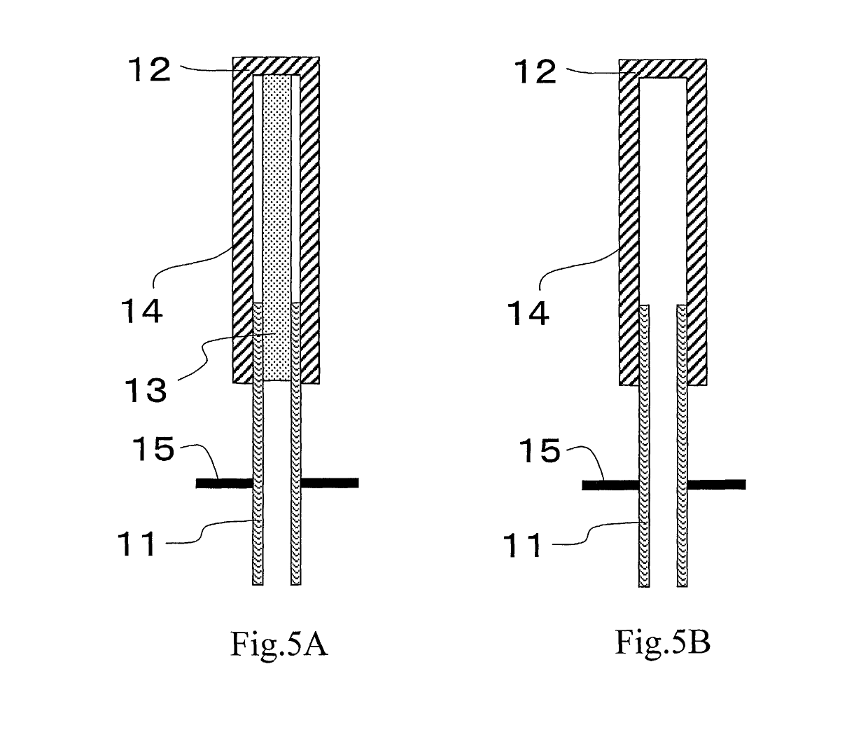

FIGS. 5A and 5B are schematic cross-sectional views for describing another example of a spotting pin according to the present invention. Illustrated therein is a spotting pin without a spring. The spotting pin shown in FIG. 5A corresponds to the spotting pin shown in FIG. 1 wherein the spring 14 is excluded therefrom. Moreover, the spotting pin shown in FIG. 5B further corresponded to the spotting pin of FIG. 5A further excluding the piston therefrom. As it has been explained with reference to FIGS. 3C and 3D, the spring 14 is required in an event of relatively modifying the external hollow tube 12 with respect to the internal hollow tube 11 for suction of the sample into the internal hollow tube 11. However, if the internal hollow tube 11 and the stopper 15 have enough weight collectively, it is possible to proceed from the state of FIG. 3C to the state of FIG. 3D to effectuate suction of the sample into the internal hollow tube 11 without any spring.

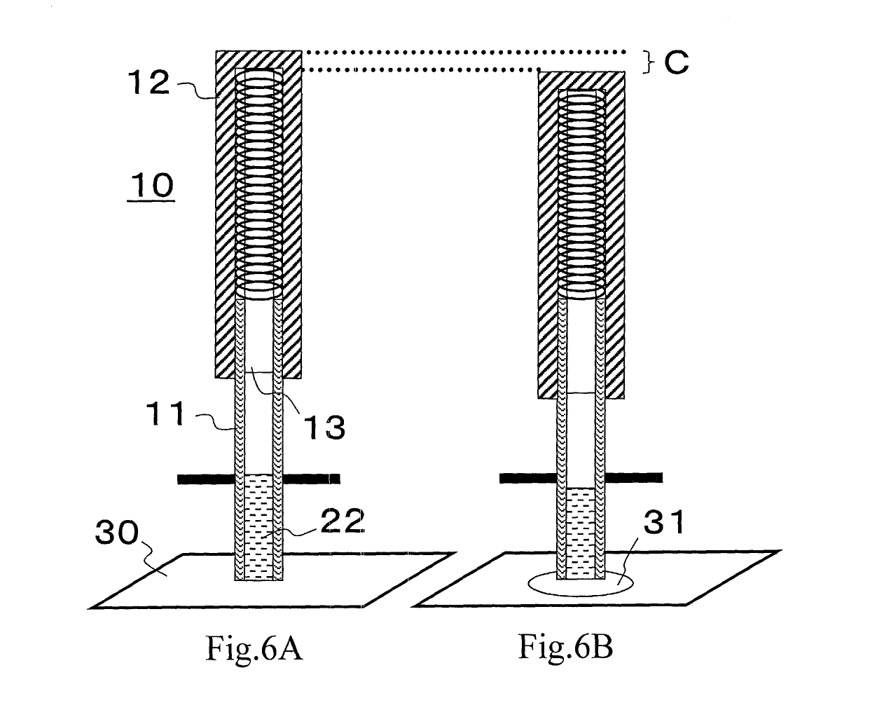

FIGS. 6A and 6B are schematic views for showing an aspect of spotting the sample 22 drawn in the internal hollow tube 11 of the spotting pin 10 onto a substrate for fabrication of a biochip such as a glass slide 30. In the state where the tip of the spotting pin 10 (the tip of the internal hollow tube 11) contacts with the surface of the glass slide 30 as shown in FIG. 6A, the external tube 12 is then pushed downward by a length C as shown in FIG. 6B. In this event, the piston 13 is also pushed toward the inside of the internal hollow tube 11 by the length C. Then, the sample 22 held in the internal hollow tube 11 is pressed by the air layer 23 (or pressed by the piston 13 touching to the sample 22 in the case of the spotting pin 10′ shown in FIGS. 4A to 4D,) so that the sample 22 flows out of the notches 16 to 19 provided on the tip of the spotting pin 10 (the tip of the internal hollow tube 11) and forms a spot 31 of the sample on the glass slide 30. In the second turn of forming a spot, the length of pushing the external tube 12 is set to 2×C; and in the nth turn of forming a spot, the length of pushing the external tube 12 is set to n×C. In this way, the sample 22 drawn in the internal hollow tube 11 of the spotting pin 10 can be spotted out on the glass slide 30 sequentially by plural times. Such spots may be formed by one per glass slide, or in plural per glass slide.

The quantity of the sample spotted on the glass slide in one operation is very small. And in general, an adhesive substance is coated on the glass slide 30, whereby the sample, which spreads circularly on the surface of the glass slide while taking the tip of the spotting pin as the center thereof, is uniformly fixed thereto by the adhesive substance. Therefore, although the use of the spotting pin of the present invention causes suction on the surface of the glass slide 30 when the sample is spotted and the spotting pin 10 is removed from the glass slide 30, such suction does not induce drawing of the spotted sample back to the spotting pin 10.

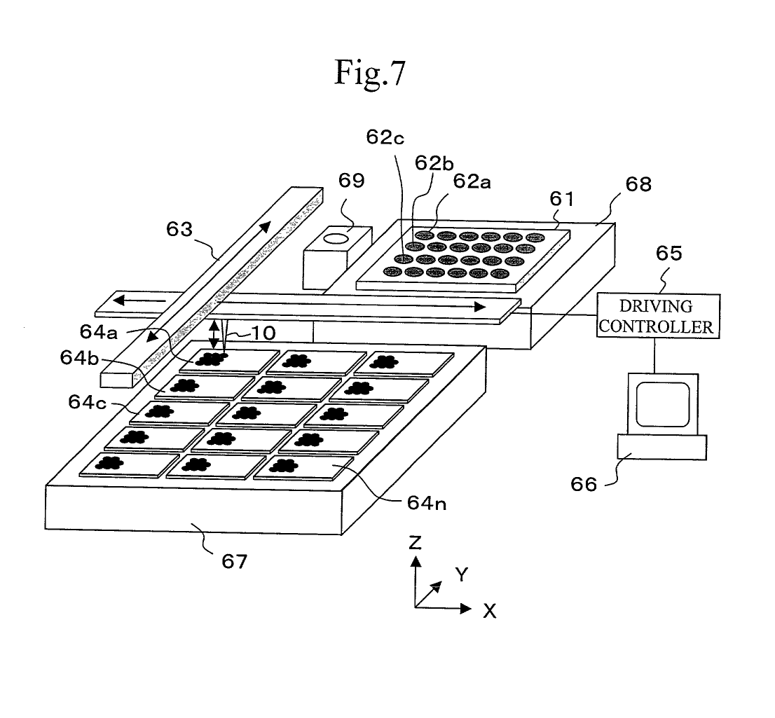

FIG. 7 is a schematic diagram of a device for fabricating biochips using the spotting pin of the present invention. The device for fabricating biochips comprises: a substrate stage 67 for placing a plurality of substrates 64a, 64b, 64c, . . . to 64n for biochips; a microplate stage 68 for placing a microplate 61 containing a plurality of samples; a cleaning tank 69 for cleaning the spotting pin; an XYZ driving unit 63 equipped with the spotting pin 10 and capable of driving a tip position of the spotting pin 10 in the X, Y and Z directions; a drive controller 65 for driving the XYZ driving unit 63; and a computer 66 for controlling the drive controller 65. The outer tube 12 of the spotting pin 10 is fixed to a Z-axis driver of the XYZ driving unit 63.

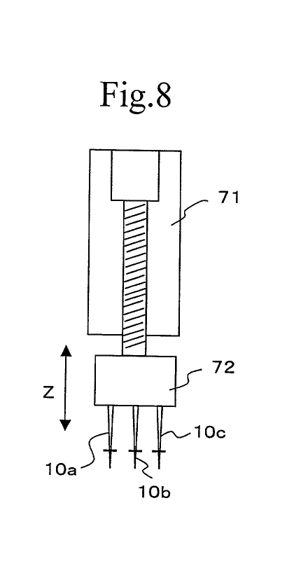

FIG. 8 is an explanatory view showing one example of the Z-axis driver 71 of the XYZ driving unit 63. In the example of FIG. 8, three spotting pins 10a, 10b and 10c are fixed by being inserted to insertion holes provided on the bottom surface of a pin head 72. The pin head 72 is accurately driven in the Z direction as indicated by an arrow by the Z-axis driver 71 under control of the drive controller 65. Although description is made herein regarding the XYZ driving unit 63 including XY rails which drive in X and Y directions and the Z-axis driver 71 which moves the pin head 72 to the Z direction, the XYZ driving unit 63 may be also constituted by a robot arm capable of three-dimensional position control.

Samples 62a, 62b, 62c and so on that contain plural types of materials such as given base sequences, respectively, are placed at given positions on the microplate 61. Moreover, various kinds of positional information are set up in the computer 66, such as positional information regarding the samples placed on the microplate 61, positional information regarding the cleaning tank 69, and information regarding positions on the substrates 64a, 64b, 64c, . . . to 64n on which the samples are to be spotted, and procedures for spotting the samples with the XYZ driving unit 63 is programmed therein.

Upon spotting the samples, the spotting pin 10 is moved to a position directly above the sample 62a on the microplate 61with the XYZ driving unit under control of the drive controller 65 controlled by the computer 66, and a given amount of the sample 62a at that position is drawn into the spotting pin 10. Thereafter, the spotting pin 10 is moved to a given position above the substrate 64a by an XY-axis driving mechanism of the XYZ driving unit 63. Then the spotting pin 10 is moved downward to the substrate 64a by a Z-axis driving mechanism of the XYZ driving unit 63, and the tip of the pin is contacted with the surface of the substrate 64a. Then, as described with FIGS. 6A and 6B, the external tube 12 of the spotting pin 10 is pushed to the Z direction by the length C to spot the sample 62a on the given position on the substrate 64a. After spotting, the Z-axis driving mechanism of the XYZ driving unit 63 pulls up the spotting pin 10, and the spotting pin 10 is moved to a given position on the adjacent substrate 64b by the XY-axis driving mechanism. Then, the external tube 12 of the spotting pin 10 is pushed to the Z direction by the length 2×C, whereby the sample 62a is spotted on the given position on the substrate 64b. Such operations are iterated with respect to the substrates 64a, 64b, 64c, . . . to 64n to spot the sample 62a sequentially. Thereafter, the spotting pin 10 is allowed to draw another sample 64b on the microplate 61 by a given amount, and the sample 64b is spotted sequentially on given positions on the substrates 64a, 64b, 64c, . . . to 64n by similar operations. By iterating such operations with respect to all the samples on the microplate 61, multiple biochips are fabricated.

Motion of the XYZ driving unit 63 to the directions of the X axis, the Y axis and the Z axis is performed by a stepping motor, for example. Output from an encoder annexed to the stepping motor is inputted to the drive controller 65. Control of XYZ positions of the spotting pin 10 is performed by the drive controller 65 by means of comparing three-dimensional coordinate positions designated by the computer 66 with current three-dimensional coordinate positions of the spotting pin 10 and by means of driving the step motor to resolve a difference therebetween to zero.

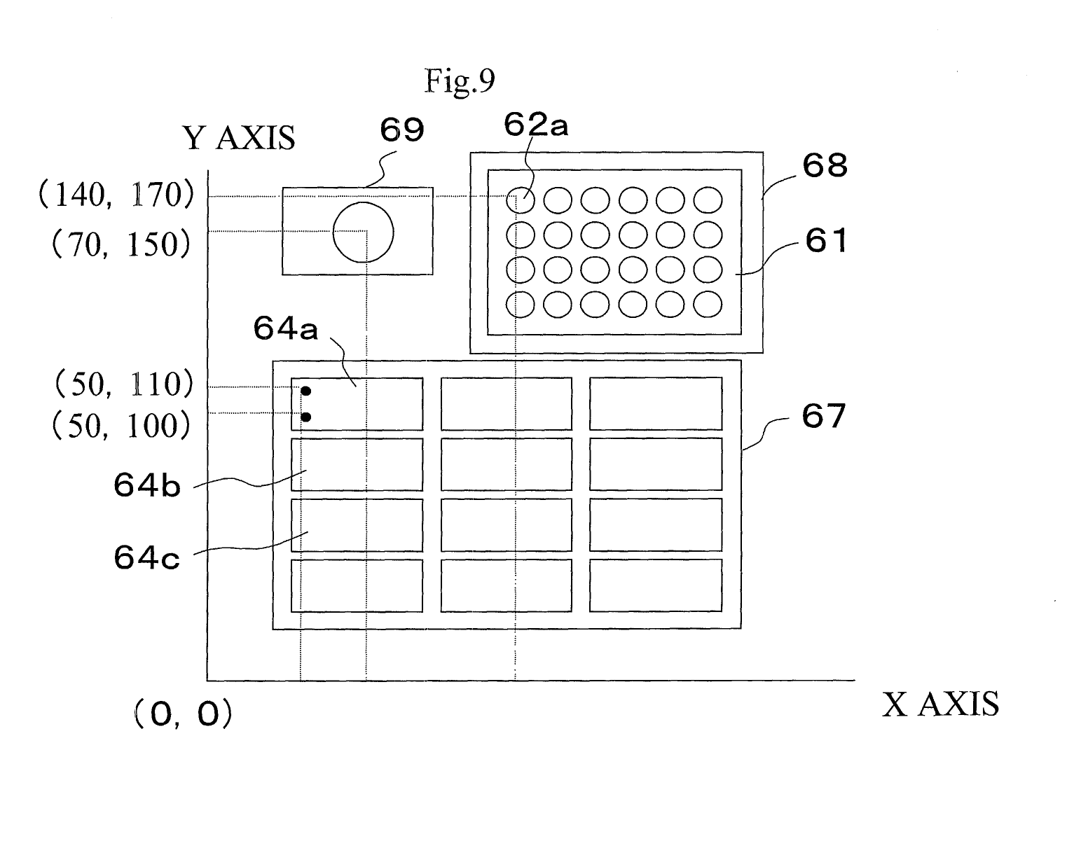

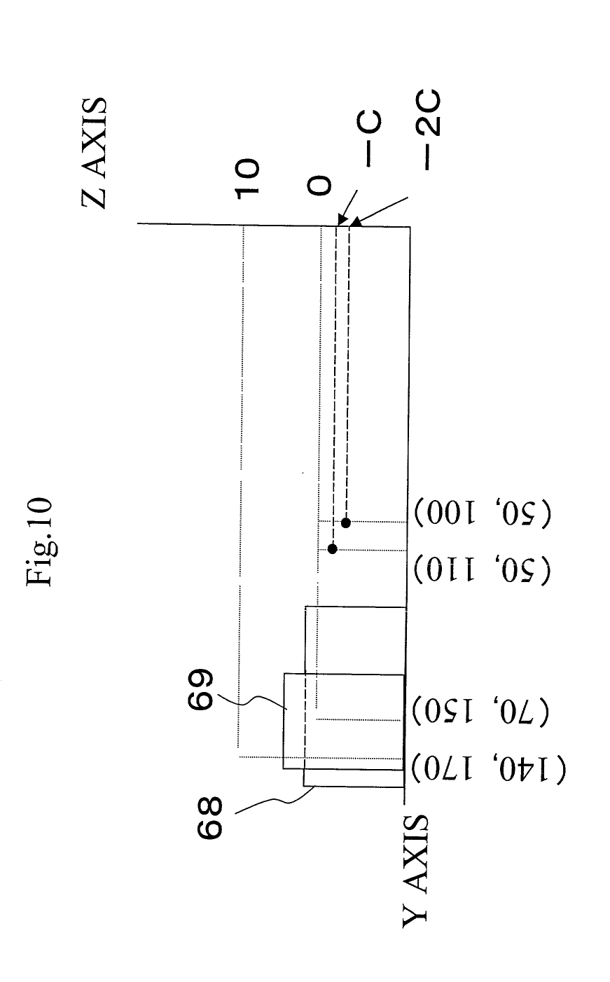

Now, description will be made regarding an example of drive control of the spotting pin 10 in the directions of the X axis, the Y axis and the Z axis by the XYZ driving unit 63, with reference to FIG. 9 and FIG. 10. FIG. 9 is a plan view taken along the X-Y plane schematically showing the microplate 61 placed on the microplate stage 68, the cleaning tank 69, and the substrates 64a, 64b, 64c, . . . to 64n for the biochips placed on the substrate stage 64. FIG. 10 is a cross-sectional view thereof taken along the Y-Z plane.

In the coordinate system illustrated in FIG. 9, coordinates (140, 170) indicate a position of a cup that contains the sample 62a, and coordinates (70, 150) indicate a position of the cleaning tank 69 for cleaning the spotting pin 10. The cleaning tank 69 is filled with a cleaning fluid for cleaning the spotting pin 10. Although twelve glass slides subject to spotting are arrayed in the drawing, the number of the glass slides is not particularly limited to twelve. The device for fabricating biochips performs, for example, drawing of the sample 62a, subsequent spotting in a position indicated by coordinates (50, 110) and second spotting in a position indicated by coordinates (50, 100).

Coordinates on the Z axis of the tip of the spotting pin 10 in the above events are illustrated with exaggeration by black circles in FIG. 10. In the event of forming a first spot, spotting is performed by setting the Z-axis coordinate of the tip of the spotting pin 10 to −C. In this case, the internal hollow tube 11 of the spotting pin 10 is pushed by a distance C. Accordingly, assuming that an area of the tip of the internal hollow tube 11 of the spotting pin 11 is S, then the sample can be spotted out by the amount expressed by S×C. Moreover, in the event of forming a second spot, spotting is performed by setting the Z-axis coordinate of the tip of the spotting pin 10 to −2×C, whereby the sample can be spotted thereon by the same amount as the first spot.

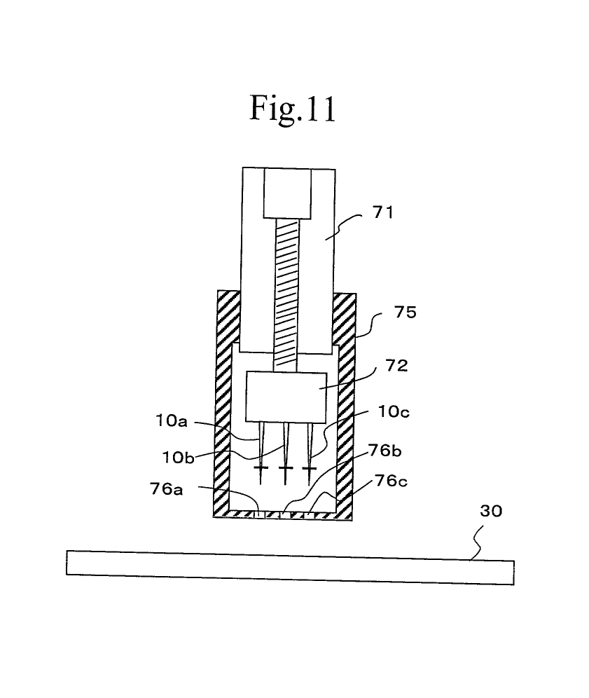

FIG. 11 is a view showing a method of spotting a sample on a substrate for a biochip while avoiding contact with the substrate. In the example as illustrated therein, a stopper abutting frame 75 is fixed to a Z-axis driver 71 of an XYZ driving unit. The stopper abutting frame 75 defines a plate member provided with orifices 76a to 76c slightly smaller than stoppers thereof, in positions corresponding to spotting pins 10a to 10c under a pin head 72.

After a sample is drawn, the above-described formation moves to a position above a substrate for a biochip, and the pin head 72 is moved downward by the Z-axis driver 71. In this event, tips of the spotting pins 10a to 10c pass through the orifices 76a to 76c of the stopper abutting frame 75, and then stoppers fixed to the tips of the pins abut on edges of the orifices 76a to 76c of the stopper abutting frame 75, whereby movement of internal hollow tubes of the spotting pins 10a to 10c is interrupted. In such a state, when the Z-axis driver 71 moves the pin head 72 further downward by a given distance, the sample is discharged from the tips of the spotting pins 10a to 10c by amounts relevant to the distance of the movement, whereby spots are formed on a substrate for a biochip placed thereunder. In this way, formation of spots becomes feasible while avoiding direct contact with the substrate.

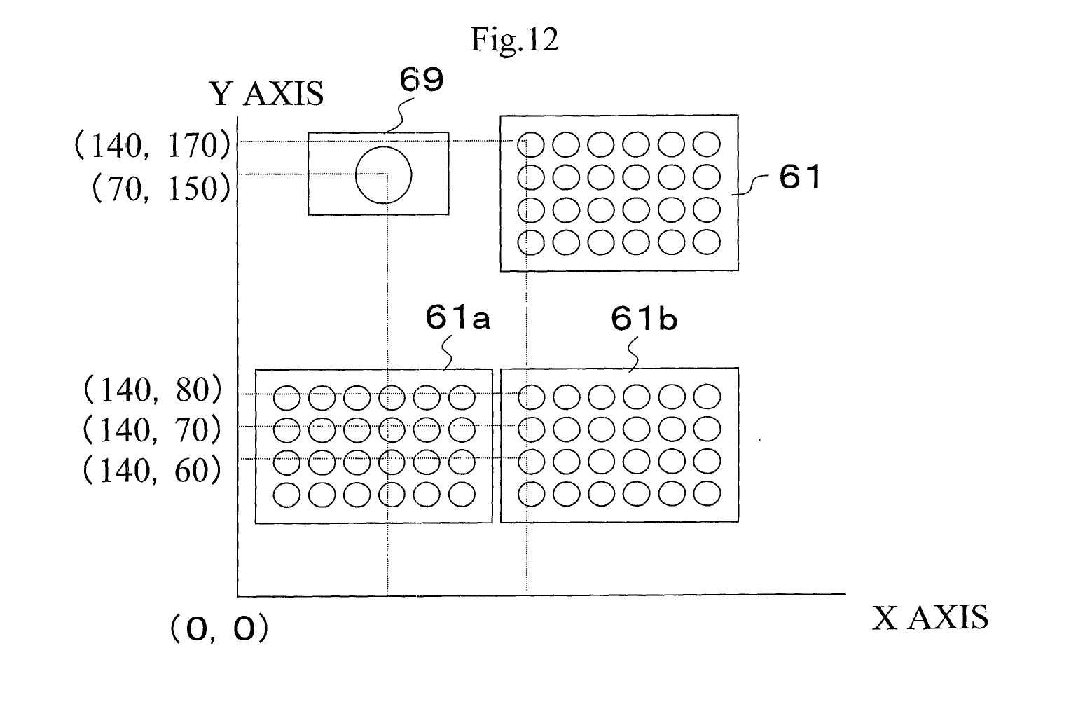

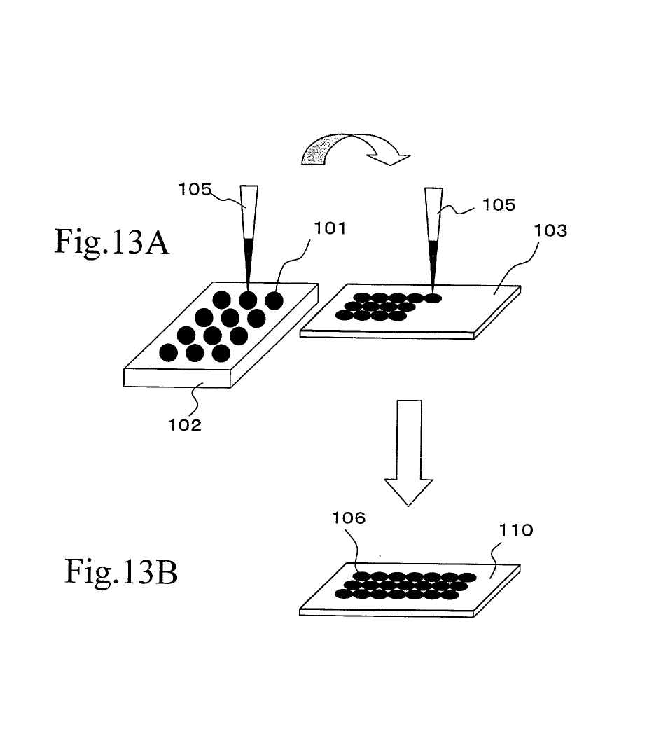

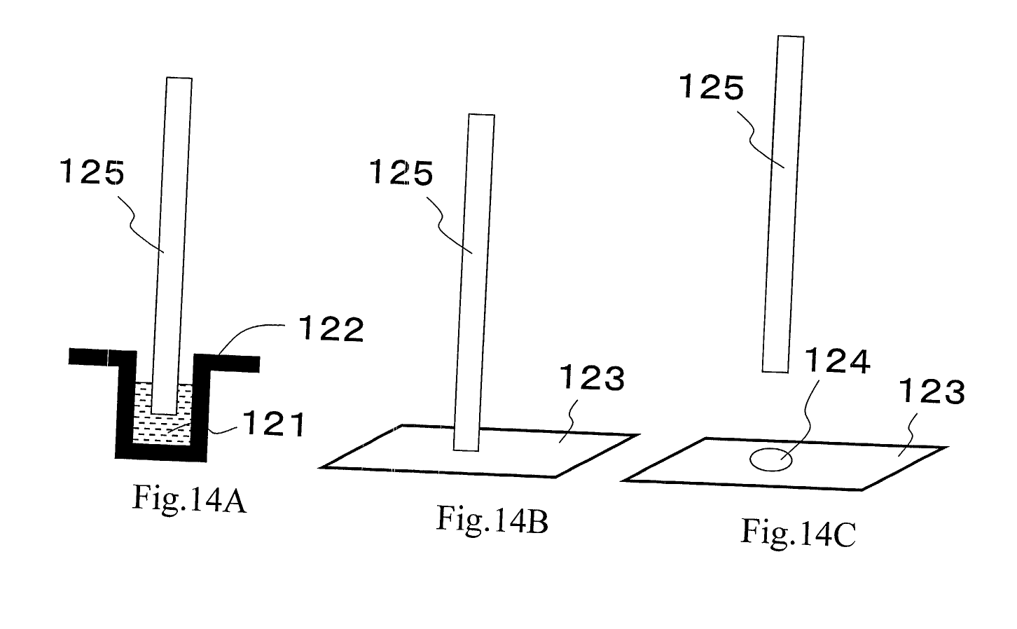

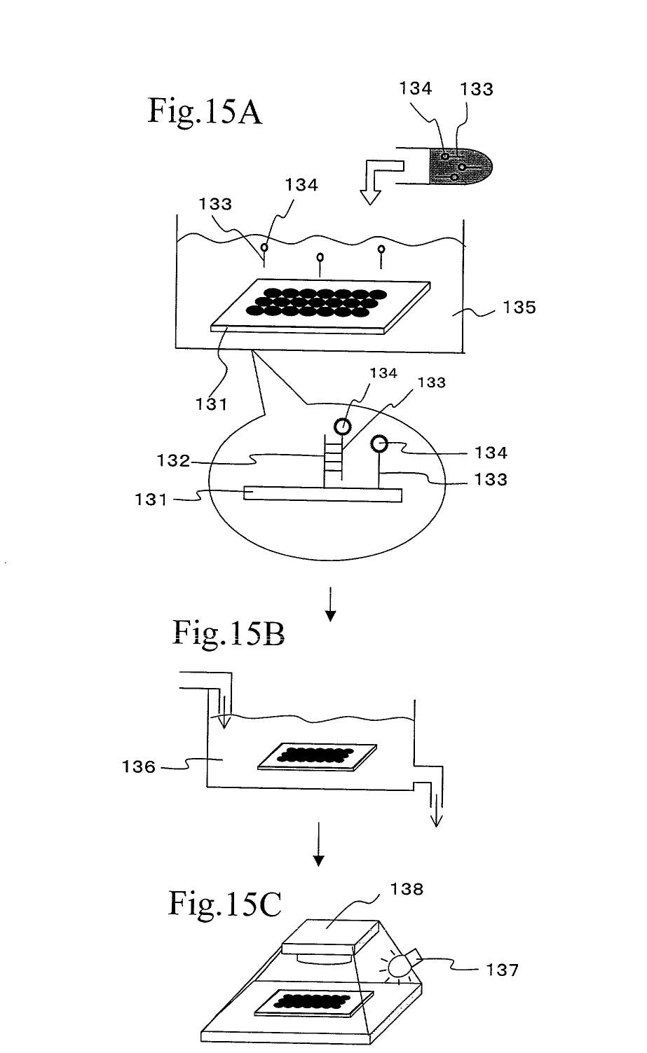

The spotting pin according to the present invention is not only usable for spotting a sample on a glass slide, but it is also usable for dispensing a sample on plates respectively provided with the same discrete sample cups. FIG. 12 is a schematic view showing an aspect of sample dispensing by use of a spotting pin of the present invention. FIG. 12 is a plan view taken along the X-Y plane, which is relevant to FIG. 9. In this event, a plurality of empty microplates 61a and 61b are placed on a biochip stage 67, instead of the substrates for biochips.

For example, a spotting pin 10 is cleaned with a cleaning tank 69 at coordinates (70, 150), and then filling of a sample is performed at coordinates (140, 170) on a microplate 61. In this case, processes for driving a pin head downward to the Z-axis direction and picking up the sample by pressing the stopper 15 of the spotting pin onto an edge of a cup on the microplate 61 are conducted in the same manner. Thereafter, the spotting pin is moved to coordinates (140, 80) and driven along the Z axis for dispensing the sample. In this event, the stopper fixed to the spotting pin 10 is abutted on an edge of a cup on the microplate 61a and a tip of the spotting pin is interrupted at a given position. Then the sample in the spotting pin 10 is discharged in accordance with a distance of movement of an external tube 12 in the Z-axis direction.

According to the present invention, time for fabricating biochips can be reduced by capability of sequential stamping. In addition, spotting samples by equal amounts on a plurality of biochips is also effectuated.

What is claimed is:

1. A spotting pin comprising:
an internal hollow tube of a tubular shape;
an external tube slidable on an outer surface of said internal hollow tube;
a piston of which one end is fixed to said external tube, and which is made slidable inside said internal hollow tube;
a spring disposed inside said external tube for resisting the force to move said external tube toward a direction of a tip of said internal hollow tube; and
a stopper provided in a position of a given distance from said tip of said internal hollow tube.

2. A spotting pin comprising:
an internal hollow tube of a tubular shape;
an external tube having a bottom, which is slidable on an outer surface of said internal hollow tube;
a spring disposed inside said external tube for resisting the force to move said external tube toward a direction of a tip of said internal hollow tube; and
a stopper provided in a position of a given distance from said tip of said internal hollow tube.

3. A spotting pin comprising:
an internal hollow tube of a tubular shape;
an external tube having a bottom, which is slidable on an outer surface of said internal hollow tube; and
a stopper provided in a position of a given distance from said tip of said internal hollow tube.

4. A spotting pin comprising:
an internal hollow tube of a tubular shape;
an external tube slidable on an outer surface of said internal hollow tube;
a piston of which one end is fixed to said external tube, and which is made slidable inside said internal hollow tube; and
a stopper provided in a position of a given distance from said tip of said internal hollow tube.

5. The spotting pin according to claim 1, wherein said internal hollow tube further includes notches on its tip.

6. The spotting pin according to claim 2, wherein said internal hollow tube further includes notches on its tip.

7. The spotting pin according to claims 3, wherein said internal hollow tube further includes notches on its tip.

8. The spotting pin according to claim 4, wherein said internal hollow tube further includes notches on its tip.

9. A device for fabricating biochips for spotting plural types of probe sequences in predetermined positions on a substrate, said device for fabricating biochips comprising:
a substrate stage for placing in alignment a plurality of substrates for fabrication of biochips thereon;
a microplate stage for placing a microplate containing plural types of probe sequences to be spotted;
an XYZ driving unit equipped with the spotting pin according to claims 1 and capable of driving a position of a tip of said spotting pin toward the X, Y and Z directions.

10. A device for fabricating biochips for spotting plural types of probe sequences in predetermined positions on a substrate, said device for fabricating biochips comprising:
a substrate stage for placing in alignment a plurality of substrates for fabrication of biochips thereon;
a microplate stage for placing a microplate containing plural types of probe sequences to be spotted;
an XYZ driving unit equipped with the spotting pin according to claims 2 and capable of driving a position of a tip of said spotting pin toward the X, Y and Z directions.

11. A device for fabricating biochips for spotting plural types of probe sequences in predetermined positions on a substrate, said device for fabricating biochips comprising:
a substrate stage for placing in alignment a plurality of substrates for fabrication of biochips thereon;
a microplate stage for placing a microplate containing plural types of probe sequences to be spotted;
an XYZ driving unit equipped with the spotting pin according to claims 3 and capable of driving a position of a tip of said spotting pin toward the X, Y and Z directions.

12. A device for fabricating biochips for spotting plural types of probe sequences in predetermined positions on a substrate, said device for fabricating biochips comprising:
a substrate stage for placing in alignment a plurality of substrates for fabrication of biochips thereon;
a microplate stage for placing a microplate containing plural types of probe sequences to be spotted;
an XYZ driving unit equipped with the spotting pin according to claims 4 and capable of driving a position of a tip of said spotting pin toward the X, Y and Z directions.

13. A device for fabricating biochips for spotting plural types of probe sequences in predetermined positions on a substrate, said device for fabricating biochips comprising:
a substrate stage for placing in alignment a plurality of substrates for fabrication of biochips thereon;
a microplate stage for placing a microplate containing plural types of probe sequences to be spotted;
an XYZ driving unit equipped with the spotting pin according to claims 5 and capable of driving a position of a tip of said spotting pin toward the X, Y and Z directions.

14. A device for fabricating biochips for spotting plural types of probe sequences in predetermined positions on a substrate, said device for fabricating biochips comprising:
a substrate stage for placing in alignment a plurality of substrates for fabrication of biochips thereon;
a microplate stage for placing a microplate containing plural types of probe sequences to be spotted;
an XYZ driving unit equipped with the spotting pin according to claims 6 and capable of driving a position of a tip of said spotting pin toward the X, Y and Z directions.

15. A device for fabricating biochips for spotting plural types of probe sequences in predetermined positions on a substrate, said device for fabricating biochips comprising:
a substrate stage for placing in alignment a plurality of substrates for fabrication of biochips thereon;
a microplate stage for placing a microplate containing plural types of probe sequences to be spotted;
an XYZ driving unit equipped with the spotting pin according to claims 7 and capable of driving a position of a tip of said spotting pin toward the X, Y and Z directions.

16. A device for fabricating biochips for spotting plural types of probe sequences in predetermined positions on a substrate, said device for fabricating biochips comprising:
a substrate stage for placing in alignment a plurality of substrates for fabrication of biochips thereon;
a microplate stage for placing a microplate containing plural types of probe sequences to be spotted;
an XYZ driving unit equipped with the spotting pin according to claims 8 and capable of driving a position of a tip of said spotting pin toward the X, Y and Z directions.
